(19) United States
(12) Patent Application Publication
(10) Pub. No.: US 2026/0041807 A1
(43) Pub. Date: Feb. 12, 2026

(54) AIR PURIFICATION DEVICE

(71) Applicant: Panasonic Intellectual Property Management Co., Ltd., Osaka (JP)

(72) Inventors: Kazuma MATSUMOTO, Aichi (JP); Hikaru SAHASHI, Aichi (JP); Yasunori ITO, Aichi (JP); Shunichiro OKABE, Kanagawa (JP); Shunsuke KORI, Aichi (JP); Ryota SAITO, Aichi (JP)

(73) Assignee: Panasonic Intellectual Property Management Co., Ltd., Osaka (JP)

(21) Appl. No.: 19/104,256

(22) PCT Filed: Jul. 4, 2023
(86) PCT No.: PCT/JP2023/024836

§ 371 Date: Feb. 17, 2025

(30) Foreign Application Priority Data
Aug. 25, 2022 (JP) 2022-133825
Aug. 25, 2022 (JP) 2022-133826

(51) Int. Cl. A61L 9/14; C02F 1/461; C02F 1/467

(52) U.S. Cl. CPC A61L 9/145; C02F 1/46104; C02F 1/4674; A61L 2209/111; A61L 2209/134; A61L 2209/213; C02F 2201/005; C02F 2201/46145; C02F 2201/4618; C02F 2201/4619; C02F 2209/40; C02F 2209/44; C02F 2303/04

(57) ABSTRACT

Air purification device includes body case, electrolytic tank, electrolysis unit, sterilization tank, gas-liquid contact portion, air blower, opening, valve member that covers opening, and movement mechanism that moves valve member. Sterilization tank is disposed below opening, by movement mechanism, when valve member is separated from an opening edge of opening, opening is opened, the water including the active oxygen species of electrolytic tank is supplied to sterilization tank, when valve member hits the opening edge of opening, opening is closed, and the supply of the water including the active oxygen species of electrolytic tank to sterilization tank is stopped.

### TECHNICAL FIELD

The present disclosure relates to an air purification device that removes bacteria, fungi, viruses, odors, and the like in air by using electrolyzed water obtained by electrifying water.

### BACKGROUND ART

A conventional air purification device includes a body case including an intake port and an outlet, a water storage container in which an electrolysis unit including electrodes for electrochemically processing water is disposed, a gas-liquid contact means for retaining water in the water storage container, an air blower for blowing air to the gas-liquid contact means, and a water supply means for automatically supplying water to make a water level of the water storage container constant. In such an air purification device, the air ventilated from the outside of the body case into the body is sterilized by passing through the gas-liquid contact means (see, for example, PTL 1).

### CITATION LIST

### Patent Literature

PTL 1: Japanese Patent No. 5994105

### SUMMARY OF THE INVENTION

In the above-described conventional air purification device, air flowing into the body passes through the gas-liquid contact means, whereby scattered substances in the air are collected into water and accumulated in the water storage container. Since most of these scattered substances accumulated in water inhibit the electrochemical process by the electrolysis unit, there is a problem that the amount of hypochlorous acid generated by the electrolysis unit decreases and the sterilization performance is not stabilized.

In order to solve this problem, it is conceivable to solve the above problem by providing an electrolytic tank disposed with an electrolysis unit and a sterilization tank including a gas-liquid contact means in a water storage container, and supplying water electrochemically processed in the electrolytic tank to the sterilization tank by a pump. In this configuration, since water is supplied from the electrolytic tank to the sterilization tank by using a pump, there is a problem that a stable sterilization and deodorization effect cannot be exhibited in a case where air locks in the pump since the amount of supplied water decreases and the amount of active oxygen species in the sterilization tank decreases.

The present disclosure has been made in view of such problems, and provides an air purification device supplying active oxygen species to a sterilization tank without excess or deficiency and exhibiting a stable sterilization effect.

An air purification device according to the present disclosure includes: a body case including an intake port and an outlet; an electrolytic tank disposed in the body case and storing water including sodium chloride; an electrolysis unit that electrolyzes the water in the electrolytic tank to generate water including active oxygen species; a sterilization tank to which the water including the active oxygen species of the electrolytic tank is supplied; a gas-liquid contact portion including a lower end immersed in the water including the active oxygen species of the sterilization tank; an air blower that blows air sucked from the intake port to the outlet via the gas-liquid contact portion; an opening located on a bottom surface of the electrolytic tank; a valve member that covers the opening; and a movement mechanism that moves the valve member, wherein the sterilization tank is disposed below the opening, and by the movement mechanism, when the valve member is separated from an opening edge of the opening, the opening is opened, and the water including the active oxygen species in the electrolytic tank is supplied to the sterilization tank, and when the valve member hits the opening edge of the opening, the opening is closed, and the supply of the water including the active oxygen species in the electrolytic tank to the sterilization tank is stopped.

According to the present disclosure, it is possible to provide an air purification device that supplies active oxygen species to a sterilization tank without excess or deficiency and exhibits a stable sterilization effect.

### BRIEF DESCRIPTION OF THE DRAWINGS

FIG. 1 is a perspective view of an air purification device according to a first exemplary embodiment of the present disclosure.

FIG. 2 is a perspective view of the air purification device in a state where a panel of the air purification device is opened.

FIG. 3 is a view showing a cross-sectional configuration when viewed from the right side when facing the front of the air purification device.

FIG. 4 is a partially exploded perspective view of the air purification device.

FIG. 5 is a partially exploded perspective view of the air purification device.

FIG. 6 is an exploded perspective view of a tank member of the air purification device.

FIG. 7 is a view showing a cross-sectional configuration of a tank member of the air purification device.

FIG. 8 is a view showing a cross-sectional configuration of a water storage container of the air purification device.

FIG. 9 is a view showing a cross-sectional configuration of a water storage container of the air purification device.

FIG. 10 is a perspective view of a water storage container of the air purification device.

FIG. 11 is a view showing a cross-sectional configuration of a water storage container of the air purification device.

FIG. 12 is a view showing a cross-sectional configuration of a water storage container of the air purification device.

FIG. 13 is an exploded perspective view of a movement mechanism of the air purification device.

FIG. 14 is a perspective view of an air purification device according to a second exemplary embodiment of the present disclosure.

FIG. 15 is a perspective view of the air purification device in a state where a panel of the air purification device is opened.

FIG. 16 is a view showing a cross-sectional configuration when viewed from the right side when facing the front of the air purification device.

FIG. 17 is a partially exploded perspective view of the air purification device.

FIG. 18 is a partially exploded perspective view of the air purification device.

FIG. 19 is an exploded perspective view of a tank member of the air purification device.

FIG. 20 is a view showing a cross-sectional configuration of a tank member of the air purification device.

FIG. 21 is a view showing a cross-sectional configuration of a water storage container of the air purification device.

FIG. 22 is a view showing a cross-sectional configuration of a water storage container of the air purification device.

FIG. 23 is a perspective view of a water storage container of the air purification device.

FIG. 24 is a view showing a cross-sectional configuration of a water storage container of the air purification device.

FIG. 25 is a view showing a cross-sectional configuration of a water storage container of the air purification device.

FIG. 26 is an exploded perspective view of a movement mechanism of the air purification device.

FIG. 27 is a plan view of a water storage container of the air purification device.

### DESCRIPTION OF EMBODIMENT

### First Exemplary Embodiment

A first exemplary embodiment of the present disclosure will be described below with reference to the accompanying drawings.

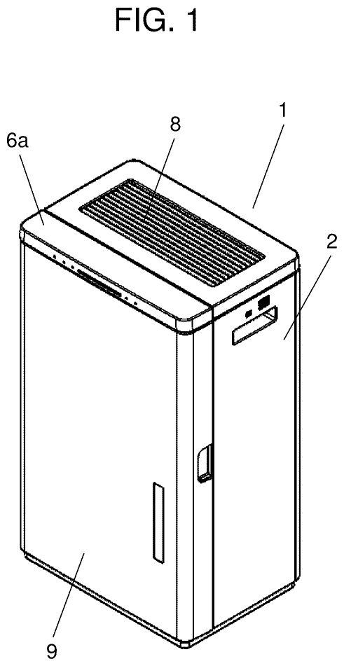

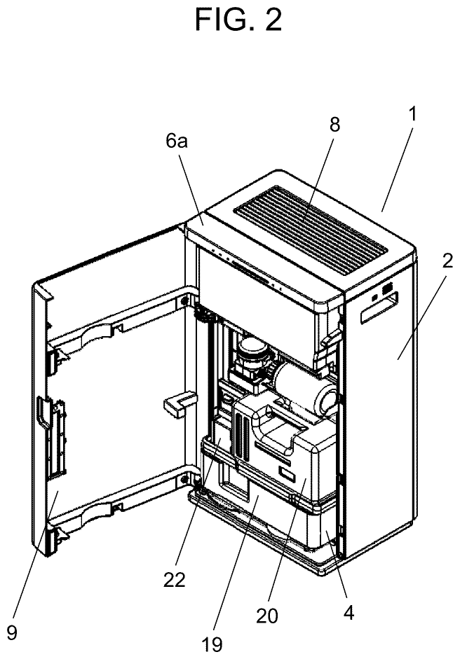

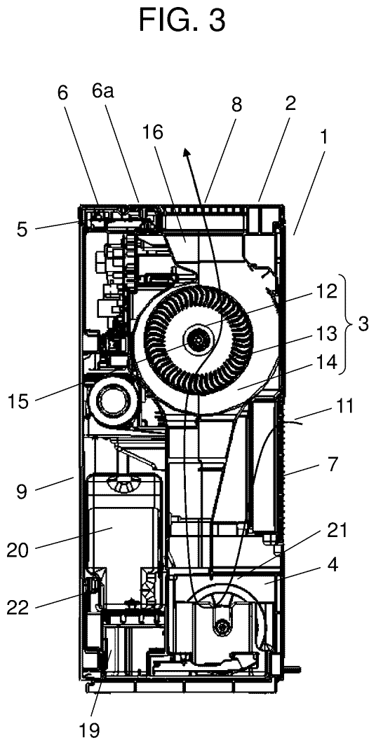

FIG. 1 and FIG. 2 are perspective views of air purification device 1 according to a first exemplary embodiment of the present disclosure. FIG. 1 is a view of the air purification device as viewed from obliquely above on the front surface side. FIG. 2 is a view of air purification device 1 with a panel opened as viewed obliquely from above on the front surface side. FIG. 3 is a view showing a cross-sectional configuration of air purification device 1 as viewed from the right side when facing the front.

Hereinafter, a detailed configuration of air purification device 1 will be described. As shown in FIG. 1 to FIG. 3, air purification device 1 of the present exemplary embodiment includes body case 2 having a substantially box shape, air blower 3, air purifier 4, controller 5, and operation part 6.

Body case 2 has a substantially vertically long box shape, and body case 2 includes intake port 7, outlet 8, and panel 9.

Intake port 7 is disposed on a back surface of body case 2.

Outlet 8 is an openable and closable type, and is disposed on a back surface side of a top surface of body case 2. Operation part 6 is disposed on a front surface side of the top surface of body case 2. Note that operation part 6 is covered with cover 6a rotatable in the vertical directions, and operation part 6 is exposed when cover 6a is opened.

Panel 9 that is openable and closable is disposed on a front surface of body case 2. In a state where panel 9 is opened, air purifier 4 is disposed in body case 2.

As illustrated in FIG. 3, in body case 2, air passage 11 (indicated by an arrow) that allows intake port 7 and outlet 8 to communicate with each other is provided. Air passage 11 includes air purifier 4 (including water storage container 19 to be described later, and a filter portion), air blower 3, and outlet 8 in this order from intake port 7.

Air blower 3 is disposed at an upper portion of body case 2 and includes motor unit 12, two fans 13 rotated by motor unit 12, and two casings 14 each having a scroll shape surrounding each of fans 13.

Motor unit 12 is a double-shaft motor, and rotating shaft 15 of motor unit 12 extends in the left-and-right direction as viewed from the front of body case 2.

Two fans 13 are sirocco fans and are fixed to both ends of rotating shaft 15 extending in the horizontal directions from motor unit 12. Motor unit 12 is disposed between fan 13 at a left side and fan 13 at a right side when viewed from the front.

Two casings 14 include discharge port 16, a first suction port (not illustrated), and a second suction port (not illustrated). Discharge port 16 is disposed on an upper surface side of body case 2 of casing 14. In addition, the first suction port is disposed on a left side surface side of body case 2 of casing 14 as viewed from the front, and the second suction port is disposed on a right side surface side of body case 2 of casing 14 as viewed from the front. When fan 13 is rotated by motor unit 12, the air outside casing 14 flows into casing 14 from the first suction port and the second suction port, and the air is blown out of casing 14 from discharge port 16.

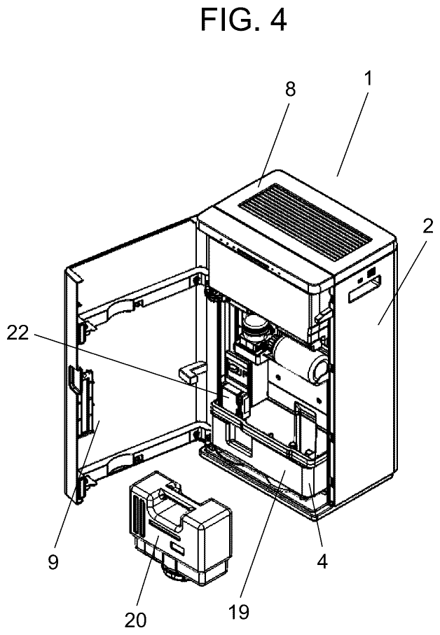

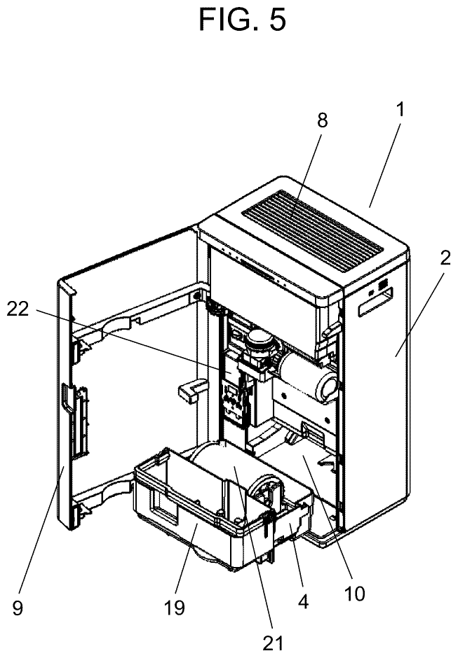

FIG. 4 is a perspective view of air purification device 1 according to the first exemplary embodiment of the present disclosure. FIG. 4 illustrates air purification device 1 viewed from obliquely above on the front surface side in a state where panel 9 is opened, and tank member 20 is taken out. FIG. 5 is a perspective view of the air purification device according to the first exemplary embodiment of the present disclosure. FIG. 5 illustrates air purification device 1 viewed from obliquely above the front surface side in a state where panel 9 is opened, and water storage container 19 is taken out.

As illustrated in FIG. 4 and FIG. 5, air purifier 4 is disposed in cavity portion 10. Cavity portion 10 is a hole recessed from the front surface side of body case 2 to the back surface side of body case 2. Air purifier 4 may be taken out from the inside of cavity portion 10 to the outside of body case 2. Air purifier 4 includes water storage container 19, tank member 20, gas-liquid contact portion 21, and electrolysis unit 22.

Water storage container 19 has a box shape having an opening on a top surface, and has a configuration of storing water. Water storage container 19 is disposed at a lower portion of body case 2, and is slidably and detachably disposed in the horizontal direction from cavity portion 10. Water storage container 19 stores water supplied from tank member 20.

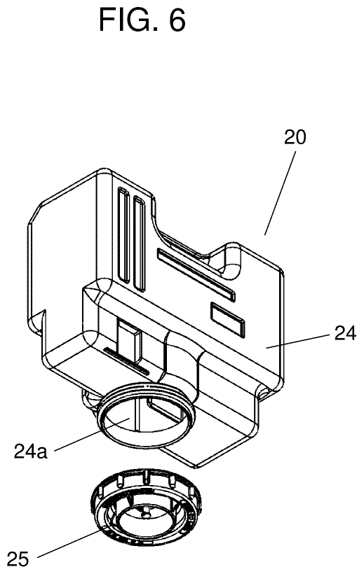

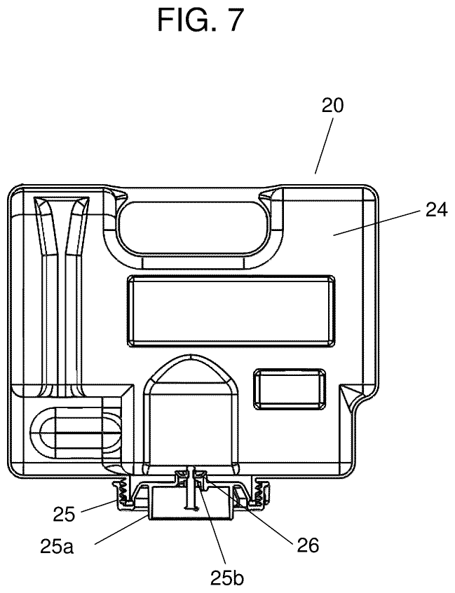

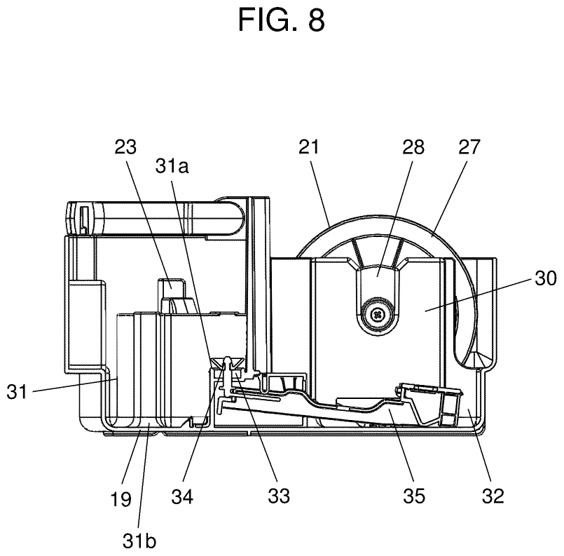

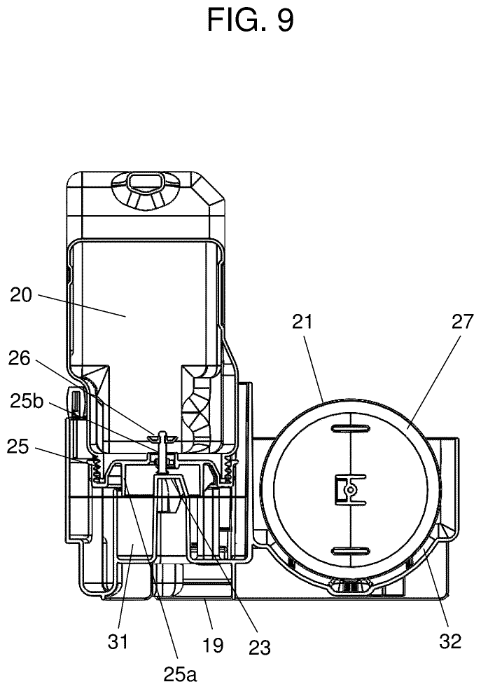

FIG. 6 is an exploded perspective view of tank member 20 according to the first exemplary embodiment of the present disclosure. FIG. 7 is a view illustrating a cross-sectional configuration of tank member 20 according to the first exemplary embodiment of the present disclosure. FIG. 8 is a view illustrating a cross-sectional configuration of water storage container 19 of the first exemplary embodiment of the present disclosure. FIG. 9 is a view illustrating a cross-sectional configuration in a state where tank member 20 and gas-liquid contact portion 21 are mounted to water storage container 19 according to the first exemplary embodiment of the present disclosure.

As illustrated in FIG. 6 to FIG. 9, tank member 20 is disposed at a lower portion of inside body case 2, and has a configuration detachable from water storage container 19. Tank holding unit 23 projecting upward is disposed on a bottom surface of water storage container 19.

Tank member 20 is mounted at a position of tank holding unit 23 disposed on a bottom surface of water storage container 19. Tank member 20 includes tank 24 that stores water, opening 24a that is disposed on the lower surface of tank 24 and communicates the inside of tank 24 and the outside of tank 24, and lid 25 that covers opening 24a.

Lid 25 includes, at the center, hole 25b communicating in the vertical directions, valve 26 that opens and closes hole 25b, and tubular portion 25a having a tubular shape surrounding hole 25b, having a central axis extending in the vertical directions, and protruding downward from lid 25. Hole 25b is located at the center of tubular portion 25a as viewed in the central axis direction.

In the above configuration, when tank member 20 is attached to the position of tank holding unit 23 of water storage container 19 with opening 24a of tank 24 facing downward, valve 26 is opened by tank holding unit 23. That is, when tank member 20 is filled with water and attached to the position of tank holding unit 23, valve 26 is moved upward by tank holding unit 23, and hole 25b of lid 25 is opened. When hole 25b of lid 25 is opened, water in tank 24 is supplied to water storage container 19 through hole 25b of lid 25, and the water is stored in water storage container 19.

When the water level in water storage container 19 rises and reaches the lower end of tubular portion 25a of lid 25, the water supply is stopped because hole 25b of tank member 20 is water-sealed. The water remains inside tank member 20, and the water inside the tank is supplied to water storage container 19 every time the water level in water storage container 19 drops. That is, the water level in water storage container 19 is kept constant.

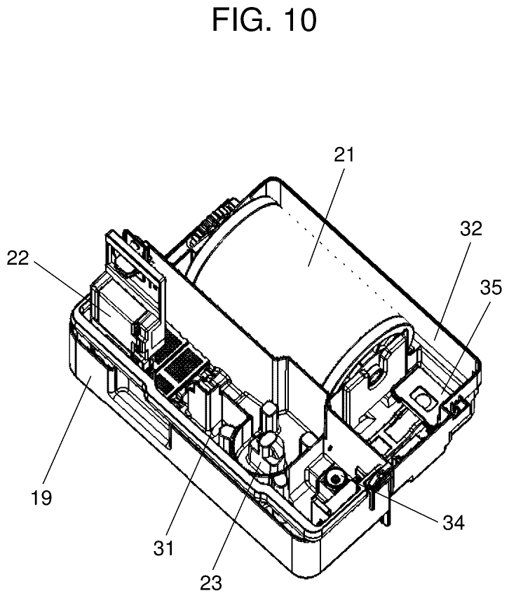

FIG. 10 is a perspective view in a case where gas-liquid contact portion 21 is mounted to water storage container 19 according to the first exemplary embodiment of the present disclosure.

As illustrated in FIG. 8 and FIG. 10, gas-liquid contact portion 21 is a member that brings water stored in water storage container 19 into contact with indoor air sucked into body case 2 by air blower 3. Gas-liquid contact portion 21 includes filter 27, filter frame 28, and a drive unit (not illustrated).

Filter 27 has a tubular shape, and has a configuration in that holes through which air flows are located in a circumferential portion. Filter 27 is mounted to filter frame 28, and one end of filter 27 is immersed in water in water storage container 19.

Filter frame 28 is rotatably supported by bearing part 30 disposed on water storage container 19. Filter 27 and filter frame 28 have a configuration that is rotated by the drive unit.

As shown in FIG. 3, FIG. 4, FIG. 5, and FIG. 10, electrolysis unit 22 is mounted in body case 2 so as to be vertically movable. FIG. 3 illustrates a state where electrolysis unit 22 has moved downward. Electrolysis unit 22 includes a first electrode (not illustrated) and a second electrode (not illustrated).

When water storage container 19 is mounted to cavity portion 10 at a lower part of body case 2 and electrolysis unit 22 is moved downward, the first electrode and the second electrode are immersed in water storage container 19 (see FIG. 3). When a voltage is applied to the first electrode and the second electrode in a state where the first electrode and the second electrode are immersed in water storage container 19, water in water storage container 19 including an electrolysis promoting solvent (not illustrated) added by a user is electrochemically processed.

An example of the electrolysis promoting solvent is sodium chloride, and hypochlorous acid is generated from the sodium chloride aqueous solution by electrolysis unit 22. Electrolysis unit 22 is detachable from body case 2.

Controller 5 controls electrolysis unit 22, a driver of gas-liquid contact portion 21, and motor unit 12 of air blower 3. Specifically, controller 5 controls a voltage to be applied to the first electrode and the second electrode of electrolysis unit 22, an operation of the driver of gas-liquid contact portion 21, a rotation speed of fan 13 of air blower 3, and the like according to an operation of operation part 6.

When fan 13 is rotated by motor unit 12 of air blower 3, air is blown into air passage 11. When fan 13 rotates, in air passage 11, the outside air that has entered body case 2 from intake port 7 is sequentially blown out of body case 2 through air purifier 4 (water storage container 19 and filter 27), air blower 3, and outlet 8.

As illustrated in FIG. 8 to FIG. 10, water storage container 19 includes electrolytic tank 31 that stores water including sodium chloride, and sterilization tank 32 to which water including active oxygen species of electrolytic tank 31 is supplied.

Electrolytic tank 31 is provided with tank member 20 and electrolysis unit 22. Tank member 20 is detachable from electrolytic tank 31 and supplies water to electrolytic tank 31. An electrolysis promoting solvent (not illustrated) is added to electrolytic tank 31 by a user, and electrolytic tank 31 stores water including sodium chloride that is an electrolysis promoting solvent.

Electrolysis unit 22 is movable in the vertical directions, and when electrolysis unit 22 is moved downward, a part of the first electrode (not illustrated) and the second electrode (not illustrated) of electrolysis unit 22 is immersed in water in electrolytic tank 31. In this state, electrolysis unit 22 electrolyzes water in electrolytic tank 31 to generate water including active oxygen species. When electrolysis unit 22 is moved upward, electrolysis unit 22 is located above water storage container 19.

In sterilization tank 32, gas-liquid contact portion 21 is disposed so that the lower end of filter 27 is immersed in water including active oxygen species. Air blower 3 blows air sucked from intake port 7 to outlet 8 via gas-liquid contact portion 21.

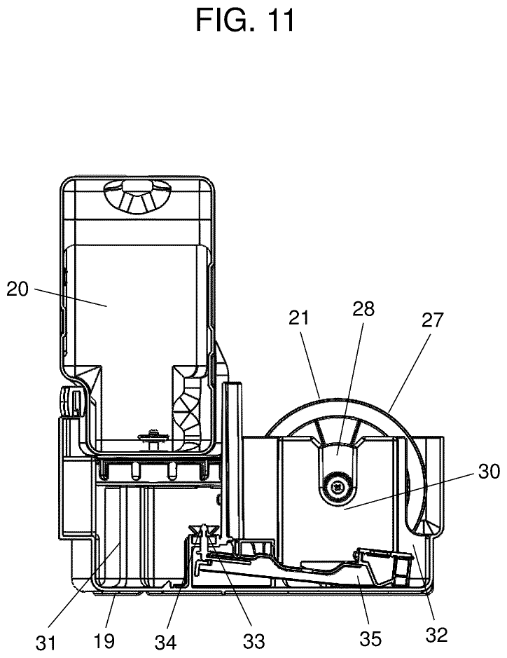

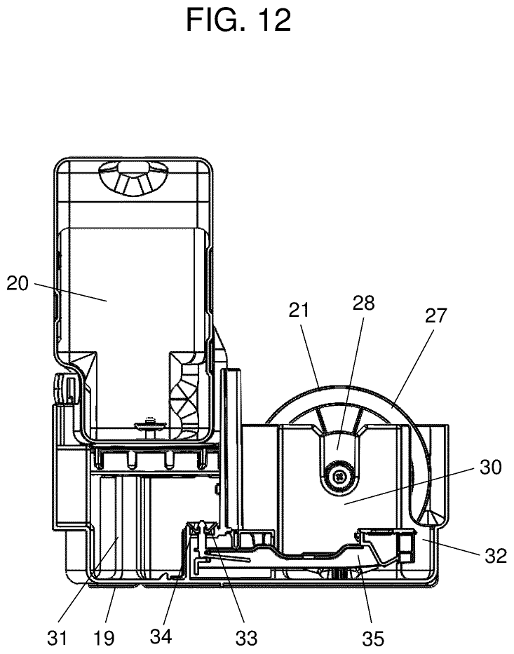

FIG. 11 is a view illustrating a cross-sectional configuration in that tank member 20 and gas-liquid contact portion 21 are mounted to water storage container 19 according to the first exemplary embodiment of the present disclosure, and illustrates a state where opening 33 is opened. FIG. 12 is a view illustrating a cross-sectional configuration in that tank member 20 and gas-liquid contact portion 21 are mounted to water storage container 19 according to the first exemplary embodiment of the present disclosure, and illustrates a state where opening 33 is closed.

As shown in FIG. 8 to FIG. 12, one of the features of the present exemplary embodiment is a configuration of supplying water including active oxygen species in electrolytic tank 31 to sterilization tank 32. Specifically, water storage container 19 includes opening 33 disposed on a bottom surface of electrolytic tank 31, valve member 34 covering opening 33, and movement mechanism 35 that moves valve member 34. A part of sterilization tank 32 is disposed below opening 33. When valve member 34 is separated from an opening edge of opening 33 by movement mechanism 35, opening 33 is opened, and water including active oxygen species in electrolytic tank 31 is supplied to sterilization tank 32. On the other hand, when valve member 34 hits the opening edge of opening 33, opening 33 is closed, and the supply of water including active oxygen species in electrolytic tank 31 to sterilization tank 32 is stopped.

In this mechanism, water flows from electrolytic tank 31 to sterilization tank 32 by opening and closing of opening 33 by valve member 34. As a result, as compared with a case where water is supplied from electrolytic tank 31 to sterilization tank 32 using a pump, there is no decrease in water supply due to air lock of the pump, active oxygen species can be stably supplied, and the supply amount can be controlled to an arbitrary amount according to its opening and closing time.

As shown in FIG. 8, the bottom surface of electrolytic tank 31 has first bottom surface 31a that is a bottom surface having opening 33, and second bottom surface 31b into which sodium chloride as an electrolysis promoting solvent is added. First bottom surface 31a is disposed above second bottom surface 31b.

As a result, it is possible to prevent sodium chloride added into second bottom surface 31b from flowing out to sterilization tank 32 before electrolysis for generating hypochlorous acid is performed.

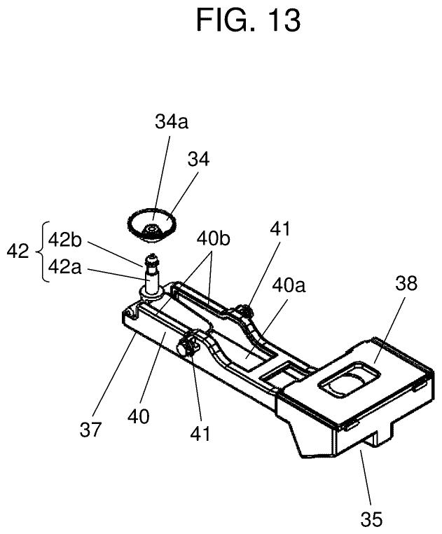

FIG. 13 is an exploded perspective view of movement mechanism 35 according to the first exemplary embodiment of the present disclosure.

As illustrated in FIG. 13, movement mechanism 35 includes lever part 37 and float portion 38.

Both end portions of lever part 37 rotate in the vertical directions. Specifically, lever part 37 includes lever portion 40, rotation shaft portion 41, and connection portion 42.

Lever portion 40 has a substantially C shape extending in the lateral direction in a side view and having a vertical cross-sectional shape opened upward, and includes lower plate 40a having a horizontally long flat plate shape and two side plates 40b having a horizontally long flat plate shape extending upward from both long sides of lower plate 40a.

Rotation shaft portion 41 is two protrusions protruding outward from two side plates 40b in a direction vertical (perpendicular) to side plates 40b and in horizontal directions. Two rotation shaft portions 41 are rotatably mounted on a lever support portion (not illustrated) disposed in water storage container 19. In lever part 37, with rotation shaft portion 41 as a rotation center, both ends of two lever portions 40 move in the vertical directions that are opposite to each other, so that one end of the two lever portions 40 moving downward when the other end moving upward.

Connection portion 42 is disposed on the other side of lever part 37 opposite to one side having float portion 38, and includes shaft portion 42a having a rod-shape protruding upward from lever part 37 and groove portion 42b having an annular shape disposed at the central portion of shaft portion 42a and recessed toward the central axis of shaft portion 42a.

Valve member 34 is made of an elastic material such as rubber. Valve member 34 has a conical shape expanding upward, and has hole 34a at the center. The opening edge of hole 34a of valve member 34 is fitted into groove portion 42b of shaft portion 42a, and valve member 34 is fixed to shaft portion 42a of connection portion 42.

Float portion 38 is disposed on the one side of lever part 37 and has buoyancy. An example of float portion 38 is a configuration having expanded polystyrene, and float portion 38 floats on the water surface of sterilization tank 32 and moves together with the water surface within the movement range of lever part 37 in the vertical directions.

In the above configuration, when the water level of sterilization tank 32 is lowered, float portion 38 is also lowered, connection portion 42 and valve member 34 are raised, valve member 34 is separated from the opening edge of opening 33, opening 33 is opened, and the water including the active oxygen species of electrolytic tank 31 is supplied to sterilization tank 32 (see FIG. 11).

On the other hand, when the water level of sterilization tank 32 rises, float portion 38 also rises, connection portion 42 and valve member 34 are lowered, valve member 34 hits the opening edge of opening 33 and closes opening 33, and the supply of water including active oxygen species in electrolytic tank 31 is stopped (see FIG. 12). As described above, the water level of sterilization tank 32 in a state where the supply of water including active oxygen species of electrolytic tank 31 to sterilization tank 32 is stopped becomes the normal sterilization tank water level.

That is, in the present disclosure, when the water level of sterilization tank 32 is lowered without using power such as electricity, valve member 34 is separated from the opening edge of opening 33, opening 33 is opened, and water flows from electrolytic tank 31 to sterilization tank 32. As a result, the water level of sterilization tank 32 rises, valve member 34 hits the opening edge of opening 33 to close opening 33, and the flow of water from electrolytic tank 31 to sterilization tank 32 is stopped. By repeating this, the water level of sterilization tank 32 is kept constant as long as water is in electrolytic tank 31.

Further, since lever part 37 is connected to valve member 34 through connection portion 42, valve member 34 also moves up and down along with the vertical movement of lever part 37. This configuration is effective in a case where a larger amount of water than expected is supplied from electrolytic tank 31 to sterilization tank 32 due to water leakage from valve member 34 occurred for some reason. Specifically, due to a large amount of water level rise in sterilization tank 32, the position of float portion 38 rises more than usual, and valve member 34 connected through connection portion 42 hits the opening edge of opening 33 with a stronger force than usual due to buoyancy of float portion 38. According to this configuration, in a case where any water leakage occurs, the water leakage is closed with a stronger force than the conventional technology, that can contribute to preventing the water leak.

Further, opening 33 disposed on the bottom surface of electrolytic tank 31 is located above the water surface in sterilization tank 32 in a state where float portion 38 is raised, connection portion 42 and valve member 34 are lowered, valve member 34 hits the opening edge of opening 33 to close opening 33, and the supply of water including active oxygen species of electrolytic tank 31 to sterilization tank 32 is stopped.

The water including active oxygen species supplied to sterilization tank 32 is vaporized by filter 27 and released to the outside of the body. At this time, components contained in the water is precipitated. If the electrolysis is continued while the components are attached to the first electrode and the second electrode of electrolysis unit 22, there is a concern that the life of electrolysis unit 22 is shortened.

In the present exemplary embodiment, opening 33 disposed on the bottom surface of electrolytic tank 31 is located above the water surface in sterilization tank 32 in a state where the supply of water including active oxygen species of electrolytic tank 31 to sterilization tank 32 is stopped. Therefore, the water including active oxygen species generated in electrolytic tank 31 is only supplied to sterilization tank 32 and does not flow back. Therefore, it is possible to reduce the occurrence of problems such as shortening the lives of the first electrode and the second electrode of electrolysis unit 22 disposed in electrolytic tank 31.

### Second Exemplary Embodiment

Hereinafter, a second exemplary embodiment of the present disclosure will be described with reference to the drawings.

In the above-described conventional air purification device, air flowing into the body passes through the gas-liquid contact means, whereby scattered substances in the air are collected into water and accumulated in the water storage container. Since most of these scattered substances accumulated in water inhibit the electrochemical process by the electrolysis unit, there is a problem that the amount of hypochlorous acid generated by the electrolysis unit decreases and the sterilization performance is not stabilized.

In order to solve this problem, it is conceivable to solve the above problem by providing an electrolytic tank including an electrolysis unit and a sterilization tank including a gas-liquid contact means in a water storage container, and providing a water level adjustment mechanism for supplying water electrochemically processed in the electrolytic tank to the sterilization tank. In this configuration, there is a problem that, in a case where an amount of water more than expected is supplied due to a failure of the water level adjustment mechanism, water overflows from the water storage container, and water leakage to the outside of the body may occur.

A second exemplary embodiment of the present disclosure has been made in view of such a problem, and provides an air purification device that exhibits stable sterilization performance and can prevent water leakage to the outside of a body case.

An air purification device according to a second exemplary embodiment of the present disclosure includes: a body case including an intake port and an outlet; an electrolytic tank disposed in the body case and storing water including sodium chloride; an electrolysis unit that electrolyzes water in the electrolytic tank, and generates water including active oxygen species; a tank member that supplies water to the electrolytic tank; a sterilization tank to which water including active oxygen species of the electrolytic tank is supplied; a gas-liquid contact portion, a lower end of the gas-liquid contact portion being immersed in water including the active oxygen species of the sterilization tank; an air blower that blows air sucked from the intake port to the outlet via the gas-liquid contact portion; an opening located on a bottom surface of the electrolytic tank; a valve member that covers the opening; and a movement mechanism that moves the valve member, wherein the sterilization tank is disposed below the opening, by the movement mechanism, when the valve member is separated from an opening edge of the opening, the opening is opened, and water including active oxygen species in the electrolytic tank is supplied to the sterilization tank, and when the valve member hits the opening edge of the opening, the opening is closed, and supply of water including the active oxygen species in the electrolytic tank to the sterilization tank is stopped, the tank member includes a water level adjustment mechanism that adjusts a water level of the electrolytic tank to a normal electrolytic tank water level, and the sterilization tank has a height of an upper end higher than a height of the normal electrolytic tank water level of the electrolytic tank.

According to the second exemplary embodiment of the present disclosure, it is possible to provide an air purification device that exhibits stable sterilization performance and can prevent water leakage to the outside of the body case.

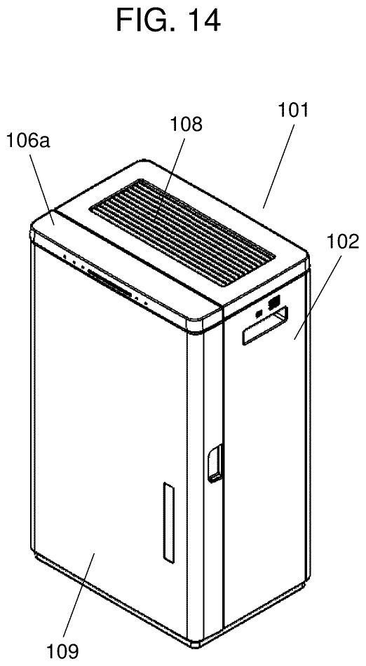

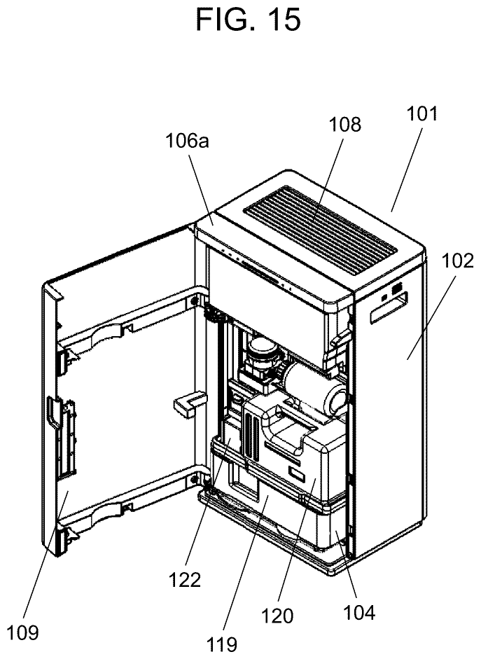

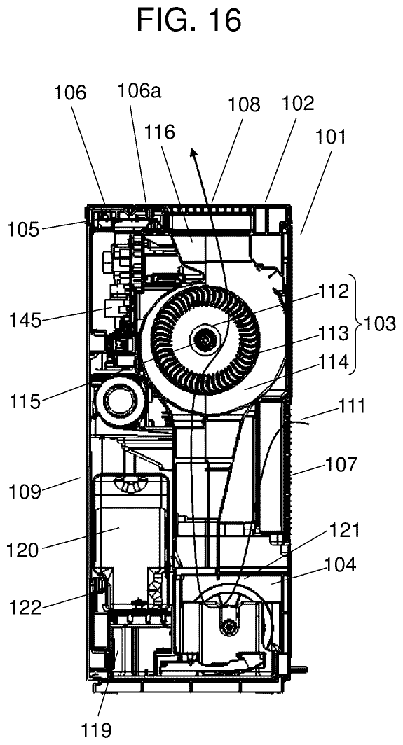

FIG. 14 and FIG. 15 are perspective views of air purification device 101 according to a second exemplary embodiment of the present disclosure. FIG. 14 is a view of air purification device 101 as viewed obliquely from above on the front surface side. FIG. 15 is a view of air purification device 101 with a panel opened as viewed obliquely from above on the front surface side. FIG. 16 is a diagram illustrating a cross-sectional configuration of air purification device 101 as viewed from the right side when facing the front.

Hereinafter, a detailed configuration of air purification device 101 will be described. As shown in FIG. 14 to FIG. 16, air purification device 101 of the present exemplary embodiment includes body case 102 having a substantially box shape, air blower 103, air purifier 104, controller 105, and operation part 106.

Body case 102 has a substantially vertically long box shape, and body case 102 includes intake port 107, outlet 108, and panel 109.

Intake port 107 is disposed on a back surface of body case 102.

Outlet 108 is an openable and closable type, and is disposed on a back surface side of a top surface of body case 102. Operation part 6 is disposed on a front surface side of the top surface of body case 102. Note that operation part 106 is covered with cover 106a rotatable in the vertical directions, and operation part 106 is exposed when cover 106a is opened.

Panel 109 that is openable and closable is disposed on a front surface of body case 102. In a state where panel 109 is opened, air purifier 104 is disposed in body case 102.

As illustrated in FIG. 16, body case 102 includes air passage 111 (indicated by an arrow) that allows intake port 107 and outlet 108 to communicate with each other. Air passage 111 includes air purifier 104 (water storage container 119 to be described later, and a filter portion), air blower 103, and outlet 108 in this order from intake port 107.

Air blower 103 is disposed at an upper portion in body case 102 and includes motor unit 112, two fans 113 rotated by motor unit 112, and two casings 114 each having a scroll shape surrounding each of fans 113.

Motor unit 112 is a double-shaft motor, and rotating shaft 115 of motor unit 112 extends in the left-and-right direction as viewed from the front of body case 102.

Two fans 113 are sirocco fans and are fixed to both ends of rotating shaft 115 extending in the horizontal directions from motor unit 112. Motor unit 112 is disposed between fan 113 at a left side and fan 113 at a right side when viewed from the front.

Two casings 114 include discharge port 116, a first suction port (not illustrated), and a second suction port (not illustrated). Discharge port 116 is disposed on an upper surface side of body case 102 of casing 114. In addition, the first suction port is disposed on a left side surface side of body case 102 of casing 114 as viewed from the front, and the second suction port is disposed on a right side surface side of body case 102 of casing 114 as viewed from the front. When fan 113 is rotated by motor unit 112, the air outside casing 114 flows into casing 114 from the first suction port and the second suction port, and the air is blown out of casing 114 from discharge port 116.

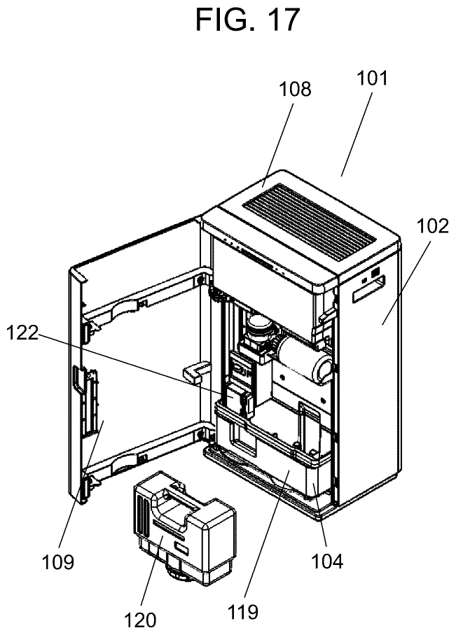

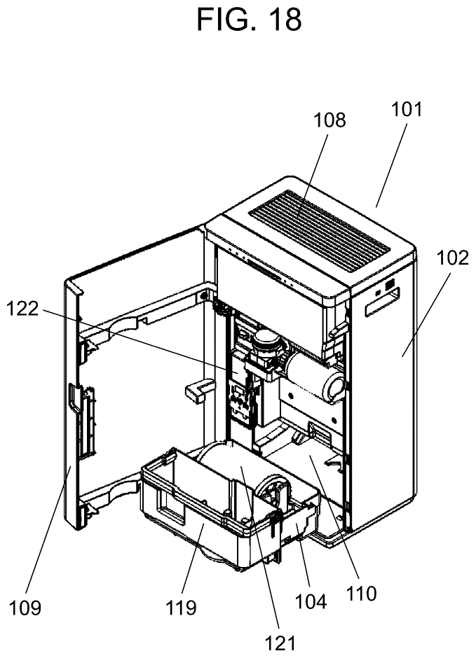

FIG. 17 is a perspective view of air purification device 101 according to the second exemplary embodiment of the present disclosure. FIG. 17 illustrates air purification device 101 viewed from obliquely above on the front surface side in a state where panel 109 is opened, and tank member 120 is taken out. FIG. 18 is a perspective view of the air purification device 101 according to the second exemplary embodiment of the present disclosure. FIG. 18 illustrates air purification device 101 viewed from obliquely above the front surface side in a state where panel 109 is opened, and water storage container 119 is taken out.

Air purifier 104 is disposed in cavity portion 110. Cavity portion 110 is a hole recessed from the front surface side of body case 102 to the back surface side of body case 102. Air purifier 104 can be taken out from the inside of cavity portion 110 to the outside of body case 102. Air purifier 104 includes water storage container 119, tank member 120, gas-liquid contact portion 121, and electrolysis unit 122.

Water storage container 119 has a box shape having an opening on a top surface, and has a configuration of storing water. Water storage container 119 is disposed at a lower portion of body case 102, and is slidably and detachably disposed in the horizontal direction from cavity portion 110. Water storage container 119 stores water supplied from tank member 120.

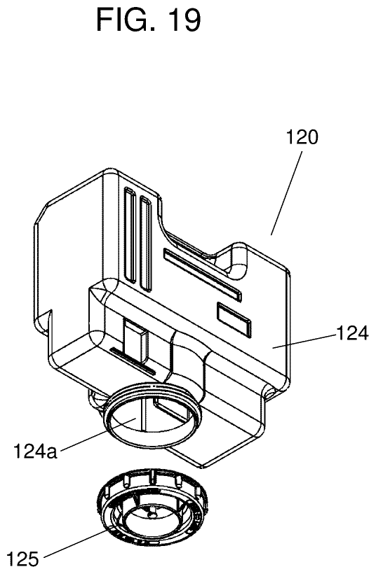

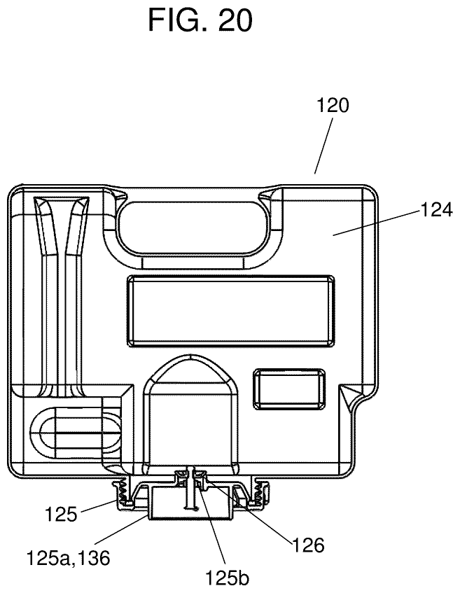

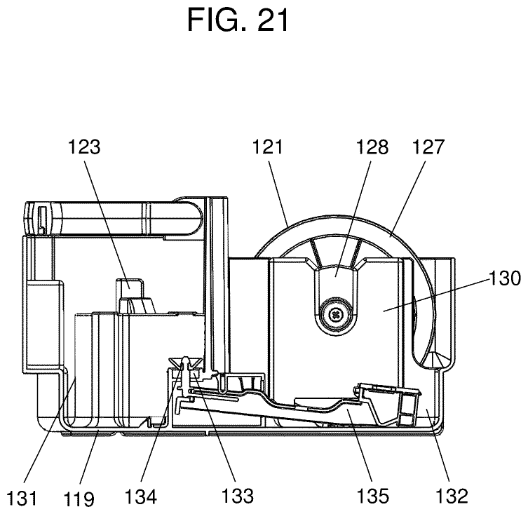

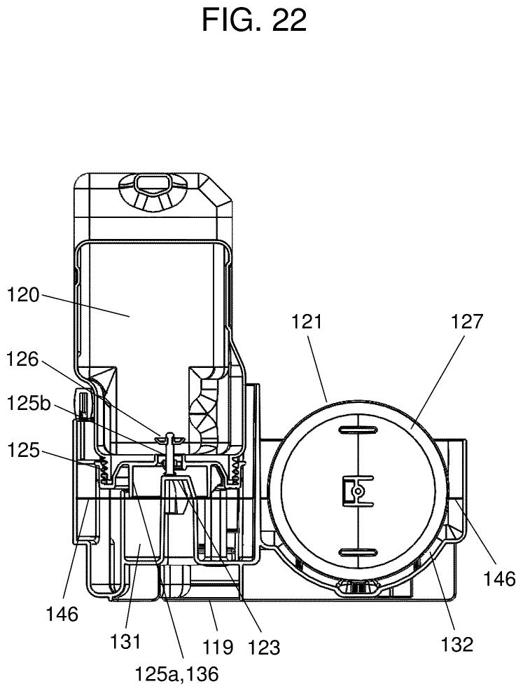

FIG. 19 is an exploded perspective view of tank member 120 according to the second exemplary embodiment of the present disclosure. FIG. 20 is a view illustrating a cross-sectional configuration of tank member 120 according to the second exemplary embodiment of the present disclosure. FIG. 21 is a view illustrating a cross-sectional configuration of water storage container 119 of the second exemplary embodiment of the present disclosure. FIG. 22 is a view illustrating a cross-sectional configuration in a state where tank member 120 and gas-liquid contact portion 121 are attached to water storage container 119 according to the second exemplary embodiment of the present disclosure.

As illustrated in FIG. 19 to FIG. 22, tank member 120 is disposed at a lower portion of inside body case 102, and has a configuration detachable from water storage container 119. Tank holding unit 123 projecting upward is disposed on a bottom surface of water storage container 119.

Tank member 120 is mounted at a position of tank holding unit 123 disposed on a bottom surface of water storage container 119. Tank member 120 includes tank 124 that stores water, opening 124a that is disposed on the lower surface of tank 124 and communicates the inside of tank 124 and the outside of tank 124, and lid 125 that covers opening 124a.

Lid 125 includes, at the center, hole 125b communicating in the vertical directions, valve 126 that opens and closes hole 125b, and tubular portion 125a having a tubular shape surrounding hole 125b, having a central axis extending in the vertical directions, and protruding downward from lid 125. Hole 125b is located at the center of tubular portion 125a as viewed in the central axis direction.

In the above configuration, when tank member 120 is attached to the position of tank holding unit 123 of water storage container 119 with opening 124a of tank 124 facing downward, valve 126 is opened by tank holding unit 123. That is, when tank member 120 is filled with water and attached to the position of tank holding unit 123, valve 126 is moved upward by tank holding unit 123, and hole 125b of lid 125 is opened. When hole 125b of lid 125 is opened, water in tank 124 is supplied to water storage container 119 through hole 125b of lid 125, and the water is stored in water storage container 119.

When the water level in water storage container 119 rises and reaches the lower end of tubular portion 125a of lid 125, the water supply is stopped because hole 125b of tank member 120 is water-sealed. The water remains inside tank member 120, and the water inside the tank is supplied to water storage container 119 every time the water level in water storage container 119 drops. That is, the water level in water storage container 119 is kept constant.

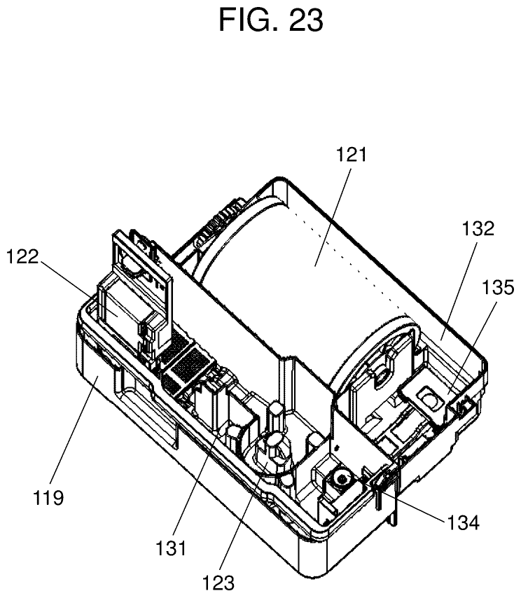

FIG. 23 is a perspective view in a case where gas-liquid contact portion 121 is attached to water storage container 119 according to the second exemplary embodiment of the present disclosure.

As illustrated in FIG. 21 and FIG. 23, gas-liquid contact portion 121 is a member that brings water stored in water storage container 119 into contact with indoor air sucked into body case 102 by air blower 103. Gas-liquid contact portion 121 includes filter 127, filter frame 128, and a drive unit (not illustrated).

Filter 127 has a tubular shape, and has a configuration in that holes through which air flows are located in a circumferential portion. Filter 127 is mounted to filter frame 128, and one end of filter 127 is immersed in water in water storage container 119.

Filter frame 128 is rotatably supported by bearing part 130 disposed on water storage container 19. Filter 127 and filter frame 128 have a configuration that is rotated by the drive unit.

As shown in FIG. 16, FIG. 17, FIG. 18, and FIG. 23, electrolysis unit 122 is mounted in body case 102 so as to be vertically movable. FIG. 16 illustrates a state where electrolysis unit 122 has moved downward. Electrolysis unit 122 has a first electrode (not illustrated) and a second electrode (not illustrated).

When water storage container 119 is mounted to cavity portion 110 at a lower part of body case 102 and electrolysis unit 122 is moved downward, the first electrode and the second electrode are immersed in water storage container 119 (see FIG. 16). When a voltage is applied to the first electrode and the second electrode in a state where the first electrode and the second electrode are immersed in water storage container 119, water in water storage container 119 including an electrolysis promoting solvent (not illustrated) added by a user is electrochemically processed.

An example of the electrolysis promoting solvent is sodium chloride, and hypochlorous acid is generated from the sodium chloride aqueous solution by electrolysis unit 122. Electrolysis unit 122 is detachable from body case 102.

Controller 105 controls electrolysis unit 122, a driver of gas-liquid contact portion 121, and motor unit 112 of air blower 103. Specifically, controller 105 controls a voltage to be applied to the first electrode and the second electrode of electrolysis unit 122, an operation of the driver of gas-liquid contact portion 121, a rotation speed of fan 113 of air blower 103, and the like according to an operation of operation part 106.

When fan 113 is rotated by motor unit 112 of air blower 103, air is blown into air passage 111. When fan 113 rotates, in air passage 111, the outside air that has entered body case 102 from intake port 107 is sequentially blown out of body case 102 through air purifier 104 (water storage container 119 and filter 127), air blower 103, and outlet 108.

As illustrated in FIG. 21 to FIG. 23, water storage container 119 includes electrolytic tank 131 that stores water including sodium chloride, and sterilization tank 132 to which water including active oxygen species of electrolytic tank 131 is supplied.

Electrolytic tank 131 includes tank member 120 and electrolysis unit 122. Tank member 120 is detachable from electrolytic tank 131 and supplies water to electrolytic tank 131. An electrolysis promoting solvent (not illustrated) is added to electrolytic tank 131 by a user, and electrolytic tank 131 stores water including sodium chloride that is an electrolysis promoting solvent.

Electrolysis unit 122 is movable in the vertical directions, and when electrolysis unit 122 is moved downward, a part of the first electrode (not illustrated) and the second electrode (not illustrated) of electrolysis unit 122 is immersed in water in electrolytic tank 131. In this state, electrolysis unit 122 electrolyzes water in electrolytic tank 131 to generate water including active oxygen species. When electrolysis unit 122 is moved upward, electrolysis unit 122 is located above water storage container 119.

In sterilization tank 132, gas-liquid contact portion 121 is disposed, and the lower end of the filter 127 is immersed in water including active oxygen species. Air blower 103 blows air sucked from intake port 107 to outlet 108 via gas-liquid contact portion 121.

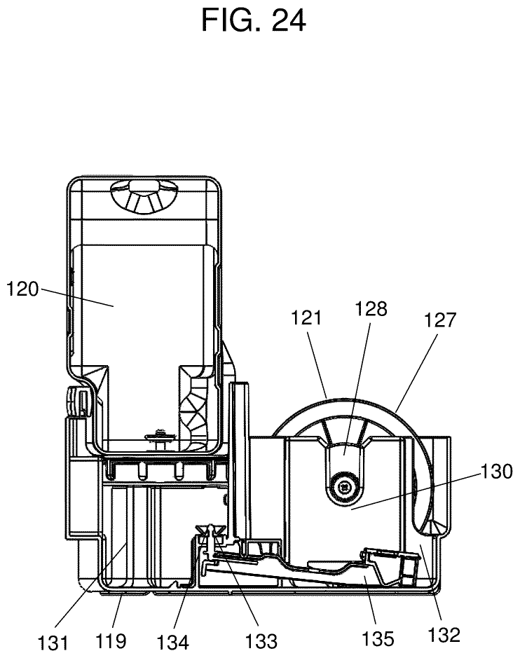

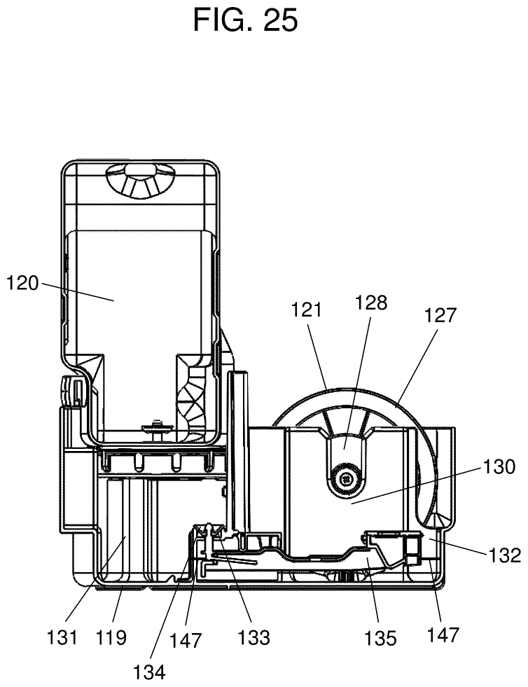

FIG. 24 is a view illustrating a cross-sectional configuration in that tank member 120 and gas-liquid contact portion 121 are mounted to water storage container 119 according to the second exemplary embodiment of the present disclosure, and illustrates a state where opening 133 is opened. FIG. 25 is a view illustrating a cross-sectional configuration in that tank member 120 and gas-liquid contact portion 121 are attached to water storage container 119 according to the second exemplary embodiment of the present disclosure, and illustrates a state in that opening 133 is closed.

As illustrated in FIG. 21 to FIG. 25, water storage container 119 includes opening 133 disposed on a bottom surface of electrolytic tank 131, valve member 134 covering opening 133, and movement mechanism 135 that moves valve member 134 in the vertical directions. Sterilization tank 132 is disposed below opening 133. When valve member 134 is separated from an opening edge of opening 133 by the movement mechanism 135, opening 133 is opened, and water including active oxygen species of electrolytic tank 131 is supplied to sterilization tank 132. On the other hand, when valve member 134 hits the opening edge of opening 133, opening 133 is closed, and the supply of water including active oxygen species in electrolytic tank 131 to sterilization tank 132 is stopped.

One of the features of the present exemplary embodiment is that tank member 120 has water level adjustment mechanism 136 that adjusts the water level of electrolytic tank 131 to normal electrolytic tank water level 146 (see FIG. 22), and the height of the upper end of sterilization tank 132 is higher than the height of the normal electrolytic tank water level 146 of electrolytic tank 131. Specifically, water level adjustment mechanism 136 is a tubular portion 125a having a tubular shape protruding downward from lid 125 of tank member 120.

In a case where water is put into tank member 120 and attached to tank holding unit 123, valve 126 is opened, water is supplied from tank member 120 to water storage container 119 through hole 125b of lid 125, and water is stored in water storage container 119. In a case where the water level in water storage container 119 rises and the water reaches the lower end of tubular portion 125a of lid 125, hole 125b of lid 125 of tank member 120 is water-sealed, and the water supply is stopped. In the present exemplary embodiment, the height of the lower end of tubular portion 125a becomes normal electrolytic tank water level 146 of electrolytic tank 131.

As a result, even in a case where movement mechanism 135 or valve member 134 fails, valve member 134 does not close opening 133, and the water continues to flow from electrolytic tank 131 to sterilization tank 132 through opening 133, it is possible to suppress overflow of the water from sterilization tank 132. That is, in a case where water to sterilization tank 132 continues to flow into sterilization tank 132 from electrolytic tank 131 through opening 133, the water level of sterilization tank 132 rises to the height of normal electrolytic tank water level 146 of electrolytic tank 131. When the water level of sterilization tank 132 rises to the height of normal electrolytic tank water level 146 of electrolytic tank 131, the water level of sterilization tank 132 and the water level of electrolytic tank 131 become the same, and the flow of water flowing from electrolytic tank 131 to sterilization tank 132 through opening 133 stops. In addition, since the height of the upper end of sterilization tank 132 having substantially a bowl-shape is higher than the height of normal electrolytic tank water level 146 of electrolytic tank 131, overflowing of water from sterilization tank 132 can be suppressed.

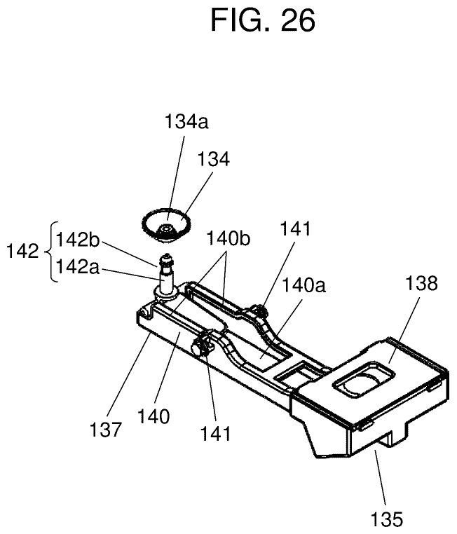

FIG. 26 is an exploded perspective view of movement mechanism 135 according to the second exemplary embodiment of the present disclosure.

As illustrated in FIG. 26, movement mechanism 135 includes lever part 137 and float portion 138.

Both end portions of lever part 137 rotate in the vertical directions. Specifically, lever part 137 includes lever portion 140, rotation shaft portion 141, and connection portion 142.

Lever portion 140 has a substantially C shape extending in the lateral direction in a side view and having a vertical cross-sectional shape opened upward, and includes lower plate 140a having a horizontally long flat plate shape and two side plates 140b having a horizontally long flat plate shape extending upward from both long sides of lower plate 140a.

Rotation shaft portion 141 is two protrusions protruding outward from two side plates 140b in a direction vertical (perpendicular) to side plates 140b and in horizontal directions. Two rotation shaft portions 141 are rotatably mounted on a lever support portion (not illustrated) disposed in water storage container 119. In lever part 137, with rotation shaft portion 141 as a rotation center, both ends of the two lever portions 140 move in the vertical directions that are opposite to each other, so that one end of the two lever portions 140 moving downward when the other end moving upward.

Connection portion 142 is disposed on the other side of lever part 137 opposite to one side having float portion 138, and includes shaft portion 142a having a rod-shape protruding upward from lever part 137 and groove portion 142b having an annular shape disposed at the central portion of shaft portion 142a and recessed toward the central axis of shaft portion 142a.

Valve member 134 is made of an elastic material such as rubber. Valve member 134 has a conical shape expanding upward, and has hole 134a at the center. The opening edge of hole 134a of valve member 134 is fitted into groove portion 142b of shaft portion 142a, and valve member 134 is fixed to shaft portion 142a of connection portion 142.

Float portion 138 is disposed on the one side of lever part 137 and has buoyancy. An example of float portion 138 is a configuration having expanded polystyrene, and float portion 138 floats on the water surface of sterilization tank 132 and moves together with the water surface within the movement range of lever part 137 in the vertical directions.

In the above configuration, when the water level of sterilization tank 132 is lowered, float portion 138 is also lowered, connection portion 142 and valve member 134 are raised, valve member 134 is separated from the opening edge of opening 133, opening 133 is opened, and the water including the active oxygen species of electrolytic tank 131 is supplied to sterilization tank 132 (see FIG. 24).

On the other hand, when the water level of sterilization tank 132 rises, float portion 138 also rises, connection portion 142 and valve member 134 are lowered, valve member 134 hits the opening edge of opening 133 and closes opening 133, and the supply of water including active oxygen species in electrolytic tank 131 is stopped (see FIG. 25). The water level of sterilization tank 132 in a state where the supply of water including active oxygen species of electrolytic tank 131 to sterilization tank 132 is stopped becomes normal sterilization tank water level 147.

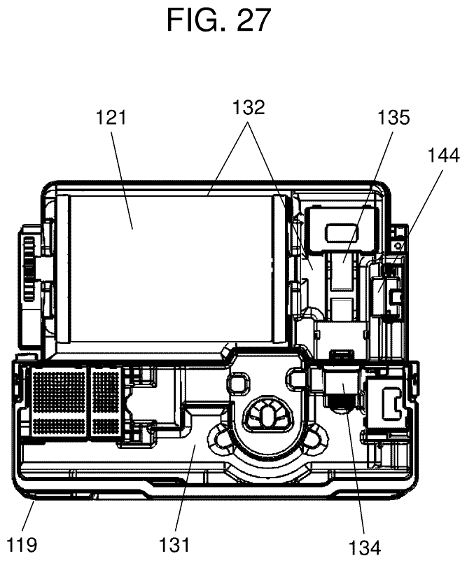

FIG. 27 is a plan view in a case where gas-liquid contact portion 121 is attached to water storage container 119 according to the second exemplary embodiment of the present disclosure.

As illustrated in FIG. 16 and FIG. 27, sterilization tank 132 is provided with water level detector 144 that detects an abnormal sterilization tank water level of sterilization tank 132. Water level detector 144 is disposed at a position higher than the height of normal sterilization tank water level 147 of sterilization tank 132 and lower than the height of normal electrolytic tank water level 146 of electrolytic tank 131. In a case where water level detector 144 detects the abnormal sterilization tank water level, notification unit 145 (see FIG. 16) that notifies a user of the abnormal sterilization tank water level is activated.

As a result, it is possible to notify the user that movement mechanism 135 or valve member 134 has failed. In a case where movement mechanism 135 or valve member 134 fails, valve member 134 does not close opening 133, and water continues to flow from electrolytic tank 131 to sterilization tank 132 through opening 133, the water level of sterilization tank 132 rises to the height of the normal electrolytic tank water level 146 of electrolytic tank 131. In a case where the water level of sterilization tank 132 reaches the abnormal sterilization tank water level before reaching normal electrolytic tank water level 146 of electrolytic tank 131, water level detector 144 detects the abnormal sterilization tank water level and notification unit 145 notifies the user. Accordingly, overflowing of water from sterilization tank 132 can be further suppressed.

In addition, movement mechanism 135 adjusts the water level of sterilization tank 132 to normal sterilization tank water level 147, and opening 133 disposed on the bottom surface of electrolytic tank 131 is located at a position higher than the water surface of normal sterilization tank water level 147.

As a result, it is possible to suppress backflow of water in sterilization tank 132 into electrolytic tank 131. Accordingly, it is possible to reduce adhesion of dirt contained in air blown into sterilization tank 132 to electrolysis unit 122 of electrolytic tank 131, and to stabilize the volatile amount of hypochlorous acid.

The present disclosure has been described above based on the exemplary embodiments. These exemplary embodiments are illustrative, and various modifications are possible for each component or combination of each processing process of the exemplary embodiments, and such modifications are also within the scope of the present disclosure.

It is also possible to combine each component or each processing process between the exemplary embodiments, and these combinations are also within the scope of the present disclosure.

### INDUSTRIAL APPLICABILITY

The present disclosure is suitable as an air purification device or the like that removes microorganisms floating in a space.

### REFERENCE MARKS IN THE DRAWINGS

1 air purification device
2 body case
3 air blower
4 air purifier
5 controller
6 operation part
6a cover
7 intake port
8 outlet
9 panel
10 cavity portion
11 air passage
12 motor unit
13 fan
14 casing
15 rotating shaft
16 discharge port
19 water storage container
20 tank member
21 gas-liquid contact portion
22 electrolysis unit
23 tank holding unit
24 tank
24a opening
25 lid
25a tubular portion
25b hole
26 valve
27 filter
28 filter frame
30 bearing part
31 electrolytic tank
31a first bottom surface
31b second bottom surface
32 sterilization tank
33 opening
34 valve member
34a hole
35 movement mechanism
37 lever part
38 float portion
40 lever portion
40a lower plate
40b side plate
41 rotation shaft portion
42 connection portion
42a shaft portion
42b groove portion
101 air purification device
102 body case
103 air blower
104 air purifier
105 controller
106 operation part
106a cover
107 intake port
108 outlet
109 panel
110 cavity portion
111 air passage
112 motor unit
113 fan
114 casing
115 rotating shaft
116 discharge port
119 water storage container
120 tank member
121 gas-liquid contact portion
122 electrolysis unit
123 tank holding unit
124 tank
124a opening
125 lid
125a tubular portion
125b hole
126 valve
127 filter
128 filter frame
130 bearing part
131 electrolytic tank
132 sterilization tank
133 opening
134 valve member
134a hole
135 movement mechanism
136 water level adjustment mechanism
137 lever part
138 float portion
140 lever portion
140a lower plate
140b side plate
141 rotation shaft portion
142 connection portion
142a shaft portion
142b groove portion
144 water level detector
145 notification unit
146 normal electrolytic tank water level
147 normal sterilization tank water level

1. An air purification device comprising:
a body case including an intake port and an outlet;
an electrolytic tank disposed in the body case and storing water including sodium chloride;
an electrolysis unit that electrolyzes the water in the electrolytic tank to generate water including active oxygen species;
a sterilization tank to which the water including the active oxygen species of the electrolytic tank is supplied;
a gas-liquid contact portion including a lower end immersed in the water including the active oxygen species of the sterilization tank;
an air blower that blows air sucked from the intake port to the outlet via the gas-liquid contact portion;
an opening located on a bottom surface of the electrolytic tank;
a valve member that covers the opening; and
a movement mechanism that moves the valve member,
wherein
the sterilization tank is disposed below the opening,
by the movement mechanism, when the valve member is separated from an opening edge of the opening, the opening is opened, and the water including the active oxygen species in the electrolytic tank is supplied to the sterilization tank, and when the valve member hits the opening edge of the opening, the opening is closed, and the supply of the water including the active oxygen species in the electrolytic tank to the sterilization tank is stopped.

2. The air purification device according to claim 1, wherein
the bottom surface of the electrolytic tank includes:
a first bottom surface that is a bottom surface having the opening; and
a second bottom surface into which sodium chloride is added, and
the first bottom surface is disposed above the second bottom surface.

3. The air purification device according to claim 1, wherein
the movement mechanism includes:
a lever part, both end portions of the lever part rotating in vertical directions;
a float portion disposed on one side of the lever part and floating on a water surface of the sterilization tank; and
a connection portion disposed on another side of the lever part and connected to the valve member,
when the water level of the sterilization tank is lowered, the float portion is lowered, the connection portion and the valve member are raised, the valve member is separated from the opening edge of the opening, the opening is opened, and the water including the active oxygen species in the electrolytic tank is supplied to the sterilization tank, and
when the water level of the sterilization tank rises, the float portion rises, the connection portion and the valve member are lowered, the valve member hits the opening edge of the opening and closes the opening, and the supply of the water including the active oxygen species of the electrolytic tank to the sterilization tank is stopped.

4. The air purification device according to claim 3, wherein the opening located on the bottom surface of the electrolytic tank is located above a water surface in the sterilization tank in a state where the float portion is raised, the connection portion and the valve member are lowered, the valve member hits the opening edge of the opening and closes the opening, and the supply of the water including the active oxygen species of the electrolytic tank to the sterilization tank is stopped.

5. The air purification device according to claim 1, further comprising a tank member that supplies the water to the electrolytic tank,
wherein the tank member includes a water level adjustment mechanism that adjusts a water level of the electrolytic tank to a normal electrolytic tank water level, and an upper end of the sterilization tank has a height higher than a height of the normal electrolytic tank water level of the electrolytic tank.

6. The air purification device according to claim 5, wherein
the tank member includes:
a tank that stores the water;
an opening located on a lower surface of the tank and communicating inside of the tank and outside of the tank; and
a lid that covers the opening,
the lid includes, at a center:
a hole communicating in vertical directions;
a valve that opens and closes the hole;
a tubular portion that is the water level adjustment mechanism and has a tubular shape surrounding the hole, having a central axis extending in vertical directions, and protruding downward from the lid, and
the hole of the tank member is water-sealed when the water level in the electrolytic tank rises and reaches a lower end of the tubular portion of the lid.

7. The air purification device according to claim 5, wherein
the movement mechanism adjusts the water level of the sterilization tank to the normal sterilization tank water level in a state where the supply of the water including active oxygen species of the electrolytic tank to the sterilization tank is stopped,
the sterilization tank includes a water level detector that detects an abnormal sterilization tank water level of the sterilization tank,
the water level detector is disposed at a position higher than the height of the normal sterilization tank water level of the sterilization tank and lower than the height of the normal electrolytic tank water level of the electrolytic tank, and
the air purification device further comprises a notification unit that notifies a user when the water level detector detects the abnormal sterilization tank water level.

8. The air purification device according to claim 7, wherein the opening located on the bottom surface of the electrolytic tank is located at a position higher than the normal sterilization tank water level of the sterilization tank.
